(19) United States
(12) Patent Application Publication
(10) Pub. No.: US 2011/0206761 A1
(43) Pub. Date: Aug. 25, 2011

(54) STABLE DOSAGE FORMS OF ANTIHYPERTENSIVE AGENTS

(76) Inventors: Nishant Babanrao Naware, Hyderabad (IN); Shailesh Suresh Bhamare, Hyderabad (IN); Prem Kumar Gidigam, Hyderabad (IN); Kishor Dattatray Deo, Hyderabad (IN); Sivakumaran Meenakshisunderam, Hyderabad (IN)

(21) Appl. No.: 12/737,964

(22) PCT Filed: Sep. 2, 2009
(86) PCT No.: PCT/IB2009/006743

§ 371 Date: May 4, 2011

(30) Foreign Application Priority Data
Sep. 4, 2008 (IN) 2157/CHE/2008

(51) Int. Cl. A61K 9/48; A61K 31/55; A61P 9/12

(52) U.S. Cl. 424/452; 514/212.07

(57) ABSTRACT

The technical field of the present invention relates to stable solid dosage form comprising combination of antihypertensive agents. More particularly, the present invention relates to stable solid dosage form comprising combination of angiotensin converting enzyme inhibitor (ACEI) or angiotensin II receptor blocker (ARB) and calcium channel blocker (CCB).

### FIELD OF THE INVENTION

The technical field of the present invention relates to stable solid dosage form comprising combination of antihypertensive agents. More particularly, the present invention relates to stable solid dosage form comprising combination of angiotensin converting enzyme inhibitor (ACEI) or angiotensin II receptor blocker (ARB) and calcium channel blocker (CCB).

### BACKGROUND OF THE INVENTION

Angiotensin converting enzyme inhibitors (ACEIs), angiotensin II receptor blockers and calcium channel blockers (CCBs) are widely used for the treatment of hypertension and various cardiovascular diseases.

Angiotensin converting enzyme inhibitors commercially available in the market include benazepril, ramipril, quinapril, enalapril, lisinopril and fosinopril, which are described in U.S. Pat. No. 4,410,520, U.S. Pat. No. 5,061,722, U.S. Pat. No. 4,344,949, U.S. Pat. No. 4,374,829, U.S. Pat. No. 4,374,829, U.S. Pat. No. 4,337,201.

ARB's act by antagonizing Angiotensin II receptor, and are therefore useful in regulating and moderating angiotensin induced hypertension, congestive heart failure, renal failure. Commercially available ARB's include valsartan marketed under the trade name Diovan®, losartan marketed under the trade name Cozaar®, irbesartan marketed under the trade name Avapro®, candesartan marketed under the trade name Atacand®, telmisartan marketed under the trade name Micardis® and eprosartan marketed under the trade name Teventen®, which are disclosed in U.S. Pat. No. 5,399,578, U.S. Pat. No. 5,138,069, U.S. Pat. No. 5,270,317, U.S. Pat. No. 5,196,444, U.S. Pat. No. 5,591,762 and U.S. Pat. No. 5,185,351.

These ACEIs and ARB's are also commercially available in combination with diuretics such as hydrochlorothiazide.

Apart from the combination of ACEIs and ARB's with diuretics, ACEIs and ARB's are also available in combination with CCB's. CCB's act by inhibiting the transmembrane influx of calcium ions into vascular smooth muscle and cardiac muscle and cause reduction in peripheral vascular resistance and reduction in blood pressure. Commercially available calcium channel blocker includes amlodipine, nifedipine, nimodipine, manidipine, nicardipine, lercanidipine, nisoldipine, felodipine and their pharmaceutically acceptable salts thereof.

Combination of CCB and ACEI provides synergistic effect in the treatment of cardiovascular disorders such as hypertension, congestive heart failure, angina, myocardial infarction, atherosclerosis, diabetic nephropathy, diabetic cardiac myopathy, renal insufficiency, peripheral vascular disease, left ventricular hypertrophy, cognitive dysfunction and stroke.

Combination of amlodipine and valsartan is being marketed as tablets under the trade name Exforge® in the United States containing 5/160, 5/320, 10/160 and 10/320 mg of amlodipine and valsartan as active ingredients and excipients such as colloidal silicon dioxide, crospovidone, magnesium stearate, microcrystalline cellulose, sodium starch glycolate, Iron oxides, hypromellose, polyethylene glycol, talc and titanium dioxide.

Fixed dose combination of amlodipine and benazepril is being marketed as capsules under the trade name Lotrel® in the United States containing 2.5/10, 5/10, 5/20, 5/40, 10/20 and 10/40 mg of amlodipine and benazepril as active ingredients and excipients such as calcium phosphate, cellulose compounds, colloidal silicon dioxide, crospovidone, gelatin, hydrogenated castor oil, iron oxides, lactose, magnesium stearate, polysorbate 80, silicon dioxide, sodium lauryl sulfate, sodium starch glycolate, starch, talc and titanium dioxide. Commercially available capsules of Lotrel contain coated compressed tablet of benazepril and amlodipine powder.

Combination of olmesartan and amlodipine is being marketed as tablets under the trade name Azor® in the United States containing 5/20, 5/40, 10/20, and 10/40 mg of amlodipine and olmesartan as active ingredients and excipients such as silicified microcrystalline cellulose, pregelatinised starch, croscarmellose sodium, magnesium stearate, polyvinyl alcohol, macrogol, titanium dioxide, talc and iron oxides.

U.S. Pat. No. 6,162,802 discloses that benazepril and amlodipine are physically incompatible substances. To overcome these problems, the inventors of US '802 developed a dosage form wherein the two active ingredients are physically separated from one another, which can be accomplished by a number of ways such as bi-layered tablets, coated pellets of one agent incorporated into a tablet of the other, separately coated pellets of each agent in a capsule or tablet, coated pellets of one agent in capsule together with powder of the other agent, each agent microencapsulated separately and then blended together for use in a tablet or capsule, use of a dual or multiple compartment transdermal device, etc.

Physical separation of two components require complicated processing and involves risks such as contamination of one drug with other drug during processing that lead to degradation. To overcome these disadvantages the following patent publications disclose different methods of stabilizing the compounds without the need of physical separation.

US 2007/0071811 discloses compositions comprising benazepril and amlodipine that are not physically separated from one another. Examples of physical contact include physical contact at an uncoated interface between tablet layers, physical contact within the fluid medium of a liquid oral or injectable dosage form, and physical contact throughout a blended mixture of the two active ingredients. US '811 exemplifies capsule dosage form prepared by wet granulating benazepril component and dry processing amlodipine component.

Similarly, US 2008/0194542 discloses stable composition consisting of benazepril and amlodipine that are not physically separated from each other in the form of uncoated tablets of one drug and powder blend of the other, uncoated granules of one drug and powder blend of the other, uncoated granules of two drugs either filled into capsules or compressed into tablets; powder blends of the two drugs filled into capsules; uncoated beads of the two drugs filled into capsules; micro-tablets of the two drugs filled into capsules.

US 2008/0096863 disclose method of stabilizing composition comprising a calcium channel blocker, an ACE inhibitor using basifying agents without the need of physical separation of the two active ingredients.

WO 2008/149201 discloses composition comprising amlodipine and benazepril and buffering agent, wherein amlodipine and benazepril are in physical contact with each other in the form of uncoated pellets of one agent incorporated into an uncoated tablet of the other, uncoated pellet or tablet of one agent together with powder or blend of the other active agent, blending a mixture of two active ingredients.

IN 1269/MUM/2007 discloses composition comprising benazepril or pharmaceutically acceptable salt thereof in combination with amlodipine or pharmaceutically acceptable salt thereof wherein at least 0.5-30% of benazepril is not physically separated from amlodipine or pharmaceutically acceptable salt thereof and at least 70-99.5% of benazepril is physically separated from amlodipine or pharmaceutically acceptable salt thereof.

Even though the above prior art references disclose different ways to achieve the stability of amlodipine and benazepril when incorporated into a single dosage form, none of the references disclose the method of stabilizing composition wherein amlodipine and benazepril are in intimate contact. However, the inventors of present invention have endeavored to develop a stable composition comprising amlodipine and benazepril using simple granulation process thus avoiding the need of physical separation of the two drugs.

### Objective of the Invention

Accordingly, the main objective of present invention is to provide stable solid dosage form of angiotensin converting enzyme inhibitor or angiotensin II receptor blocker and calcium channel blocker, which comply with the reference product in terms of in vivo parameters like Cₘₐₓ, tₘₐₓ and AUC and in vitro parameters like dissolution, disintegration.

Yet another objective of the present invention is to provide simple and efficient process for preparing stable solid dosage form of angiotensin converting enzyme inhibitor or angiotensin II receptor blocker and calcium channel blocker on a commercial scale.

### SUMMARY OF THE INVENTION

Accordingly, the present invention provides a stable solid dosage form comprising combination of antihypertensive agents which are in intimate contact to each other comprising:

i) intragranular portion comprising about 5% to 40% w/w of angiotensin converting enzyme inhibitor (ACEI) or angiotensin II receptor blocker (ARB), about 1% to 10% w/w of calcium channel blocker (CCB), about 40% to 80% w/w of diluent and
ii) extragranular portion comprising about 1% to 5% w/w of disintegrant, about 0.5% to 5% w/w of glidant.

### DETAILED DESCRIPTION OF THE INVENTION

In another embodiment, angiotensin converting enzyme inhibitor includes ramipril, benazepril, captopril, enalapril, quinapril, perindopril, lisinopril, fosinopril, trandolapril, moexipril and their pharmaceutically acceptable salts thereof.

In another embodiment, angiotensin II receptor blocker includes valsartan, losartan, irbesartan, telmisartan, candesartan, eprosartan and olmesartan and their pharmaceutically acceptable salts thereof.

In another embodiment, calcium channel blocker includes amlodipine, nifedipine, nimodipine, manidipine, nicardipine, lercanidipine, nisoldipine, felodipine and their pharmaceutically acceptable salts thereof.

The ratio of angiotensin converting enzyme inhibitor (ACEI) or angiotensin II receptor blocker (ARB) and calcium channel blocker (CCB) is from about 2.5:1 to about 8:1.

In another embodiment, wherein calcium channel blocker is present in an amount of about 1% to 20% w/w; angiotensin converting enzyme inhibitor is present in an amount of about 5% to 40% w/w and angiotensin II receptor blocker is present in an amount of about 5% to 40% w/w.

The intragranular portion further comprises one or more excipients selected from binders, disintegrants, surfactants, glidants and the like.

The extragranular portion further comprises one or more excipients selected from diluents, lubricants and the like.

The amount of intragranular portion ranges from about 80% to 97% by weight and extragranular portion ranges from about 3% to 20% by weight of composition.

Suitable diluents used according to the present invention are selected from sucrose, dextrose, lactose, mannitol, sorbitol, starch, microcrystalline cellulose, silicified microcrystalline cellulose and the like or combination thereof.

Suitable binders used according to the present invention are selected from the group comprising of hydroxypropylmethylcellulose, maize starch, povidone, hydroxypropylcellulose, pregelatinized starch and the like or combination thereof.

Suitable disintegrants used according to the present invention are selected from starch, crospovidone, sodium starch glycolate, croscarmellose sodium and the like or combination thereof.

The surfactant of the present invention may be selected from anionic or non-ionic surfactants such as sodium lauryl sulphate, polyethylene-propylene glycol copolymer (poloxamer), polyoxyethylene stearates (Macrogol-stearate), polysorbates, propylene glycol, and the like or mixtures thereof.

Suitable glidants of the present invention include magnesium silicate, talc, colloidal silicon dioxide, starch and the like.

Suitable lubricants used according to the present invention are selected from magnesium stearate, hydrogenated castor oil, calcium stearate, sodium stearyl fumerate, talc, vegetable oils, stearic acid, fumaric acid, glyceryl behenate and the like.

In another embodiment, the stable solid dosage forms of the present invention are prepared either by dry granulation or wet granulation.

The dosage form of present invention is stable at accelerated conditions. The main degradation product of benazepril is (s,s)-diacid (benazeprilat), which is the active metabolite of benazepril. The main degradation product of amlodipine is impurity D (-ethyl 5-methyl 2-[(2-aminoethoxy)methyl]-4-(2-chlorophenyl)-6-methylpyridine-3,5-dicarboxylate).

The dosage form when stored for about 3 months at about 40° C. and about 75% relative humidity, contains not more than 2.0% of (s,s)-diacid by weight relative to the benazepril and/or not more than 0.3% of impurity D by weight relative to the amlodipine.

In another embodiment, there is provided a process for the preparation of a stable solid dosage form comprising combination of antihypertensive agents which are in intimate contact to each other comprising:

i) intragranular portion comprising about 5% to 40% w/w of angiotensin converting enzyme inhibitor (ACEI) or angiotensin II receptor blocker (ARB), about 1% to 10% w/w of calcium channel blocker (CCB), about 40% to 80% w/w of diluent and
ii) extragranular portion comprising about 1% to 5% w/w of disintegrant, about 0.5% to 5% w/w of glidant, comprising the steps of:
i) granulating angiotensin converting enzyme inhibitor or angiotensin II receptor blocker and calcium channel blocker, diluent and one or more intragranular excipients using aqueous/nonaqueous binder solution,
ii) drying the granules of step (i) and
iii) blending the dried granules of step (ii) with extragranular excipients and
iv) compressing the blend into tablets or filling into capsules.

The solvents used for granulation include methylene chloride, isopropyl alcohol, acetone, methanol, ethanol, water, and the like or mixtures thereof.

In another embodiment, there is provided an alternate process for the preparation of a stable solid dosage form comprising combination of antihypertensive agents which are in intimate contact to each other comprising:

i) intragranular portion comprising about 5% to 40% w/w of angiotensin converting enzyme inhibitor (ACEI) or angiotensin II receptor blocker (ARB), about 1% to 10% w/w of calcium channel blocker (CCB), about 40% to 80% w/w of diluent and
ii) extragranular portion comprising about 1% to 5% w/w of disintegrant, about 0.5% to 5% w/w of glidant, comprising the steps of:
i) sifting and blending angiotensin converting enzyme inhibitor or angiotensin II receptor blocker and calcium channel blocker, diluent and one or more intragranular excipients,
ii) subjecting the blend to compaction/slugging to form coprimates,
ii) converting the coprimates to form granules,
iv) blending the granules with extragranular excipients and
v) compressing the granules of step (iv) to form the tablets or filling into capsules.

In a preferred embodiment, a stable solid dosage form comprising combination of antihypertensive agents which are in intimate contact to each other comprising:

i) intragranular portion comprising about 5% to 40% w/w of benazepril hydrochloride, about 1% to 10% w/w of amlodipine besylate, about 40% to 80% w/w of diluent and
ii) extragranular portion comprising about 1% to 5% w/w of disintegrant, about 0.5% to 5% w/w of glidant.

In another preferred embodiment, the stable solid dosage form comprising combination of antihypertensive agents which are in intimate contact to each other comprises:

i) intragranular portion comprising about 5% to 40% w/w of angiotensin converting enzyme inhibitor or angiotensin II receptor blocker, about 1% to 10% w/w of calcium channel blocker, about 40% to 80% w/w of diluent, about 1% to 10% w/w of disintegrant, about 0.5% to 5.0% w/w of binder and optionally about 0.1% to 5% w/w of surfactant and,
ii) extragranular portion comprising about 1% to 5% w/w of disintegrant, about 0.5% to 5% w/w of glidant and about 0.5% to 5% w/w of lubricant.

In another preferred embodiment, the stable solid dosage form comprising combination of antihypertensive agents which are in intimate contact to each other comprises:

i) intragranular portion comprising about 5% to 40% w/w of benazepril hydrochloride, about 1% to 10% w/w of amlodipine besylate, about 40% to 80% w/w of diluent selected from lactose, starch, microcrystalline cellulose or combination thereof; about 1% to 10% w/w of disintegrant selected from starch, crospovidone, sodium starch glycolate or combination thereof; about 0.5% to 5.0% w/w of binder selected from povidone, pregelatinised starch and optionally 0.1% to 5% w/w of surfactant selected from sodium lauryl sulphate, polysorbates and
ii) extragranular portion comprising about 1% to 5% w/w of disintegrant starch, crospovidone, sodium starch glycolate or combination thereof; about 0.5% to 5% w/w of glidant selected from colloidal silicon dioxide or talc and about 0.5% to 5% w/w of lubricant selected from hydrogenated castor oil or magnesium stearate.

In a preferred embodiment, the stable solid dosage forms comprising combination of antihypertensive agents which are in intimate contact to each other are prepared by a process, which comprises the steps of:

(i) granulating about 5% to 40% w/w of benazepril hydrochloride, about 1% to 10% w/w of amlodipine besylate, about 10% to 75% w/w of diluent, about 1% to 10% w/w of disintegrant and optionally about 0.1% to 5% w/w of surfactant using about 0.5% to 5.0% w/w of aqueous binder solution,
(ii) drying the granules of step (i),
(iii) blending the dried granules with about 1% to 5% w/w of disintegrant, about 0.5% to 5% w/w of glidant and about 0.5% to 5% w/w of lubricant and
(iv) formulating the granules obtained in step (iii) into solid dosage form.

In another embodiment, the solid dosage forms include tablets and capsules. The tablets may be uncoated or optionally coated.

In another embodiment, the solid dosage form is a capsule comprising the granules encapsulated in a shell.

In yet another embodiment, the present invention also provides a method of treating hypertension, congestive heart failure, angina, myocardial infarction, atherosclerosis, diabetic nephropathy, diabetic cardiac myopathy, renal insufficiency, peripheral vascular disease, left ventricular hypertrophy, cognitive dysfunction, stroke and headache by administering solid dosage form of the present invention.

The following examples further exemplify the invention and are not intended to limit the scope of the invention. It is obvious to those skilled in the art to find out the composition for other dosage forms and substitute the equivalent excipients as described in this specification or with the one known to the industry.

### Example 1

| | | Qty per unit |
|---|---|---|
| S. No. | Ingredients | (mg) |
| | Intra granular ingredients | |
| 1 | Benazepril HCl | 40.000 |
| 2 | Amlodipine besylate | 13.888 |
| 3 | Lactose anhydrous | 159.582 |
| 4 | Microcrystalline cellulose | 139.530 |
| 5 | Crospovidone | 14.000 |
| 6 | Povidone | 13.000 |
| 7 | Purified water | Q.S |
| 8 | Extragranular ingredients | |
| 9 | Crospovidone | 12.000 |
| 10 | Colloidal silicon dioxide | 4.000 |
| 11 | Hydrogenated castor oil | 4.000 |

The processing steps involved in manufacturing benazepril and amlodipine dosage form given in example 1 are given below:

i) benazepril hydrochloride, amlodipine besylate, lactose, microcrystalline cellulose, crospovidone, were sifted and blended,
ii) aqueous binder solution of povidone was prepared,
iii) granulated the blended material of step (i) with binder solution of step (ii),
iv) dried the granules obtained in step (iii) and blended with extragranular crospovidone and colloidal silicon dioxide,
v) lubricated the blend of step (iv) with hydrogenated castor oil and
vi) the lubricated blend was compressed to obtain tablets or filled into capsules.

### Example 2

| | | Qty per unit |
|---|---|---|
| S. No. | Ingredients | (mg) |
| | Intra granular ingredients | |
| 1 | Benazepril HCl | 40.000 |
| 2 | Amlodipine besylate | 13.888 |
| 3 | Lactose monohydrate | 138.112 |
| 4 | Microcrystalline cellulose | 90.000 |
| 5 | Pregelatinized starch | 44.000 |
| 6 | Maize starch | 23.400 |
| 7 | Crospovidone | 14.000 |
| 8 | Polysorbate 80 | 1.600 |
| 9 | Povidone | 13.000 |
| 10 | Purified water | Q.S |
| | Extragranular ingredients | |
| 11 | Sodium starch glycolate | 12.000 |
| 12 | Colloidal silicon dioxide | 4.000 |
| 13 | Magnesium stearate | 6.000 |

The processing steps involved in manufacturing benazepril and amlodipine dosage form given in example 2 are given below:
i) benazepril hydrochloride, amlodipine besylate, lactose, microcrystalline cellulose, crospovidone, were sifted and blended,
ii) binder solution of povidone, polysorbate 80 in water was prepared,
iii) granulated the blended material of step (i) with binder solution of step (ii),
iv) dried the granules obtained in step (iii) and blended with extragranular crospovidone and colloidal silicon dioxide,
v) lubricated the blend of step (iv) with hydrogenated castor oil and
vi) the lubricated blend was compressed to obtain tablets or filled into capsules.

### Example 3

| | | Qty per unit |
|---|---|---|
| S. No. | Ingredients | (mg) |
| | Intra granular ingredients | |
| 1 | Benazepril HCl | 10.00 |
| 2 | Amlodipine besylate | 3.47 |
| 3 | Lactose monohydrate | 225.00 |
| 4 | Microcrystalline cellulose | 139.53 |
| 5 | Crospovidone | 14.00 |
| 6 | Povidone | 13.00 |
| 7 | Isopropyl alcohol | Q.S |
| | Extragranular ingredients | |
| 8 | Crospovidone | 12.00 |
| 9 | Colloidal silicon dioxide | 4.00 |
| 10 | Magnesium stearate | 4.00 |

The processing steps involved in manufacturing benazepril and amlodipine dosage form given in example 3 are given below:
i) benazepril hydrochloride, amlodipine besylate, lactose, microcrystalline cellulose, crospovidone, were sifted and blended,
ii) a binder solution of polyvinylpyrrolidone in isopropyl alcohol was prepared,
iii) granulated the blended material of step (i) with binder solution of step (ii),
iv) dried the granules obtained in step (iii) and blended with extragranular crospovidone and colloidal silicon dioxide,
v) lubricated the blend of step (iv) with magnesium stearate and
vi) the lubricated blend was compressed to obtain tablets or filled into capsules.
The compositions described in examples 4 and 5 were prepared using the procedure similar to the one described in example 3.

### Example 4

| | | Qty per unit |
|---|---|---|
| S. No. | Ingredients | (mg) |
| | Intra granular ingredients | |
| 1 | Benazepril HCl | 10.00 |
| 2 | Amlodipine besylate | 6.95 |
| 3 | Lactose monohydrate | 221.52 |
| 4 | Microcrystalline cellulose | 139.53 |
| 5 | Crospovidone | 14.00 |
| 6 | Povidone | 13.00 |
| 7 | Isopropyl alcohol | Q.S |
| | Extragranular ingredients | |
| 8 | Crospovidone | 12.00 |
| 9 | Colloidal silicon dioxide | 4.00 |
| 10 | Magnesium stearate | 4.00 |

### Example 5

| | | Qty per unit |
|---|---|---|
| S. No. | Ingredients | (mg) |
| | Intra granular ingredients | |
| 1 | Benazepril HCl | 40.00 |
| 2 | Amlodipine besylate | 6.95 |
| 3 | Lactose monohydrate | 191.52 |
| 4 | Microcrystalline cellulose | 139.53 |
| 5 | Crospovidone | 14.00 |
| 6 | Povidone | 13.00 |
| 7 | Isopropyl alcohol | Q.S |
| | Extragranular ingredients | |
| 8 | Crospovidone | 12.00 |
| 9 | Colloidal silicon dioxide | 4.00 |
| 10 | Magnesium stearate | 4.00 |

### Stability Study of Dosage Form Containing Amlodipine and Benazepril

The dosage forms prepared according to present invention were subjected to stability study for 3 months at 40° C. and 75% relative humidity. Samples were analyzed by HPLC method and the results are summarized in table 1.

### Dissolution Profile of Capsule Formulation Containing Amlodipine and Benazepril

The dissolution profile of the amlodipine and benazepril capsules prepared according to the present invention were carried out in 900 ml of 0.01N hydrochloric acid as medium according to the procedure described in the USP, Apparatus USP I/900 ml, Basket, @ 75 rpm speed. The release profile (% of drug released in minutes) is given in table 2.

TABLE 1
Stability data of amlodipine and benazepril capsules

| | Example 3 | | Example 4 | | Example 5 | |
|---|---|---|---|---|---|---|
| Impurity | Initial | 3 month | Initial | 3 month | Initial | 3 month |
| Impurity D of | 0 | 0 | 0 | 0 | 0 | 0 |
| Amlodipine | | | | | | |
| s, s diacid | 0.16 | 1.62 | 0.12 | 1.58 | 0.1 | 1.31 |
| Total impurities | 0 | 2.94 | 0.66 | 2.76 | 0.81 | 2.22 |

TABLE 2
Dissolution profile of amlodipine and benazepril capsules

| Time in | Example 3 | | Example 4 | | Example 5 | |
|---|---|---|---|---|---|---|
| minutes | Amlodipine | Benazepril | Amlodipine | Benazepril | Amlodipine | Benazepril |
| 5 | 77 | 79 | 88 | 89 | 81 | 81 |
| 10 | 98 | 100 | 100 | 101 | 97 | 98 |
| 15 | 98 | 101 | 101 | 102 | 97 | 98 |
| 30 | 98 | 101 | 101 | 102 | 97 | 99 |

We claim:

1. A stable solid dosage form comprising combination of antihypertensive agents which are in intimate contact to each other comprising:
(i) intragranular portion comprising about 5% to 40% w/w of benazepril hydrochloride, about 1% to 10% w/w amlodipine besylate, about 40% to 80% w/w of diluent, and
(ii) extragranular portion comprising about 1% to 5% w/w of disintegrant, about 0.5% to 5% w/w of glidant.

2. The dosage form of claim 1, wherein the intragranular portion further comprise one or more excipients selected from binder, disintegrant, surfactant, glidant.

3. The dosage form of claim 1, wherein the extragranular portion further comprise diluent and lubricant.

4. The dosage form of claim 1, wherein the diluent is selected from sucrose, dextrose, lactose, mannitol, sorbitol, starch, microcrystalline cellulose, silicified microcrystalline cellulose or combination thereof.

5. The dosage form of claim 1, wherein the disintegrant is selected from starch, crospovidone, sodium starch glycolate, croscarmellose sodium.

6. The dosage form of claim 1, wherein the glidant is selected from magnesium silicate, talc, colloidal silicon dioxide, starch.

7. The dosage form of claim 2, wherein the binder is selected from hydroxypropylmethylcellulose, maize starch, povidone, hydroxypropylmethylcellulose, pregelatinized starch.

8. The dosage form of claim 3, wherein the lubricant is selected from magnesium stearate, hydrogenated castor oil, calcium stearate, sodium stearyl fumarate, talc, vegetable oils, stearic acid, fumaric acid, glyceryl behenate.

9. A stable solid dosage form comprising combination of antihypertensive agents which are in intimate contact to each other comprising:
(i) intragranular portion comprising about 5% to 40% w/w of angiotensin converting enzyme inhibitor (ACEI) or angiotensin II receptor blocker (ARB), about 1% to 10% w/w of calcium channel blocker (CCB), about 40% to 80% w/w of diluent, and
(ii) extragranular portion comprising about 1% to 5% w/w of disintegrant, about 0.5% to 5% w/w of glidant.

10. A process for the preparation of a stable solid dosage form comprising combination of antihypertensive agents which are in intimate contact to each other comprising:
(i) intragranular portion comprising about 5% to 40% w/w of angiotensin converting enzyme inhibitor (ACEI) or angiotensin II receptor blocker (ARB), about 1% to 10% w/w of calcium channel blocker (CCB), about 40% to 80% w/w of diluent, and
(ii) extragranular portion comprising about 1% to 5% w/w of disintegrant, about 0.5% to 5% w/w of glidant, comprising the steps of:
(i) granulating angiotensin converting enzyme inhibitor or angiotensin II receptor blocker and calcium channel blocker, diluent and one or more intragranular excipients using aqueous/nonaqueous binder solution,
(ii) drying the granules of step (i)
(iii) blending the dried granules of step (ii) with extragranular excipients, and
(iv) compressing the blend into tablets or filling into capsules

11. The dosage form of claim 10, wherein the solvent used for granulation is selected from methylene chloride, isopropyl alcohol, acetone, methanol, ethanol, water or mixture thereof.

12. (canceled)

13. (canceled)

14. A stable dosage form comprising combination of antihypertensive agents which are in intimate contact to each other comprising:
i) intragranular portion comprising about 5% to 40% w/w of angiotensin converting enzyme inhibitor or angiotensin II receptor blocker, about 1% to 10% w/w of calcium channel blocker, about 40% to 80% w/w of diluent, about 1% to 10% w/w of disintegrant, about 0.5% to 5.0% of w/w of binder and optionally 0.1% to about 5% w/w of surfactant, and
ii) extragranular portion comprising about 1% to 5% w/w of disintegrant, about 0.5% to 5% w/w of glidant and about 0.5% to 5% w/w of lubricant.

15. A stable dosage form comprising combination of antihypertensive agents which are in intimate contact to each other comprising:
iii) intragranular portion comprising about 5% to 40% w/w of benazepril hydrochloride, about 1% to 10% w/w of amlodipine besylate, about 40% to 80% w/w of diluent selected from lactose, starch microcrystalline cellulose or combination thereof; about 1% to 10% w/w of disintegrant selected from starch, crospovidone, sodium starch glycolate or combination thereof; about 0.5% to 5.0% of w/w of binder selected from povidone, pregelatinised starch, and
iv) extragranular portion comprising about 1% to 5% w/w of disintegrant selected from starch, crospovidone, sodium starch glycolate or combination thereof; about 0.5% to 5% w/w of glidant selected from colloidal silicon dioxide or talc and about 0.5% to 5% w/w of lubricant selected from hydrogenate castor oil or magnesium stearate.

16. A method of treating hypertension, congestive heart failure, angina, myocardial infarction, atherosclerosis, diabetic nephropathy, diabetic cardiac myopathy, renal insufficiency, peripheral vascular disease, left ventricular hypertrophy, cognitive dysfunction, stroke and headache by administering stable dosage forms of claim 1.
